(19) United States
(12) Patent Application Publication
(10) Pub. No.: US 2023/0099877 A1
(43) Pub. Date: Mar. 30, 2023

(54) RELEASE OF ACTIVE INGREDIENTS USING SILICON-CONTAINING POLYMERS

(71) Applicant: WACKER CHEMIE AG, Munich (DE)

(72) Inventor: Elke FRITZ-LANGHALS, Ottobrunn (DE)

(73) Assignee: WACKER CHEMIE AG, Munich (DE)

(21) Appl. No.: 16/613,883

(22) PCT Filed: May 16, 2017
(86) PCT No.: PCT/EP2017/061696

§ 371 Date: Nov. 15, 2019

(51) Int. Cl. A61K 9/14; A61K 47/34; C08L 83/08

(52) U.S. Cl. CPC A61K 9/146; A61K 47/34; C08L 83/08

(57) ABSTRACT

Active substances for which controlled release is desired, are released by a process in which a shaped body (S) containing a composition (C) comprises a silicone-containing polymer (P) containing at least one Si—C bonded amino acid group and active substance (A) is contacted with water, resulting in the release of active substance (A) from the composition (C).

### CROSS REFERENCE TO BELATED APPLICATIONS

This application is the U.S. National Phase of PCT Appln. No. PCT/EP2017/061696 filed May 16, 2017, the disclosure of which is incorporated in its entirety by reference herein.

### BACKGROUND OF THE INVENTION

### 1. Field of the Invention

The invention relates to an active substance (A) for use in a process for its release, in which a shaped body (S) containing a composition (C) that comprises silicone-containing polymer (P) and active substance (A) is admixed with water, resulting in the release of active substance (A) from the composition (C); to the shaped body (S); and to a process for the production of the shaped bodies (S).

### 2. Description of the Related Art

Active substances are used in the most diverse fields, particularly in medicine and pharmacy, in cosmetics, in agriculture, and in biotechnology.

The action of these substances is known to be strongly dependent on the dose. If the dose is too high, an intensification of undesirable side effects often occurs. If the dose is too low, the action is weak or absent altogether.

With direct administration of the active substance, the active substance, concentration is initially often undesirably high, but the concentration then swiftly decreases as a consequence of subsequent processes, for example through metabolic processes or dilution. For these reasons, it is of great benefit if active substances can be released as evenly as possible over a longer period of time. This approach is also known as “controlled release.” A further positive aspect of even active substance release is that this allows active substances to be used considerably more efficiently and avoids excessive consumption.

Matrices used for the controlled release of active substances include polymer materials from which the active substance may be released.

What is often desirable in such a case is for the controlled release of active substances to take place through contact with damp or water-containing media, for example in soil, in the case of agricultural uses, or, in pharmacy and medicine, in the body through contact with body fluids or on the surface of the skin through sweat. The release of the active substance depends on permeability of the poly met to water in a damp or aqueous environment (for an overview see R. Po, J. Macromolec. Sc. C, Rev. Macromol. Chem. Phys., C34, S. 650, 1994). Examples of polymers used are carboxymethylcellulose or copolymers derived from sodium acrylate and starch. However, a disadvantage of these polymer materials is that they gradually dissolve during the swelling process. What would therefore be desirable is a material that is largely insoluble in water and does not have the above disadvantages.

Polymers based on silicone have to date been used occasionally as matrices for active substances. Advantages of silicone-containing polymers are their high biocompatibility, their chemical inertness, and their insolubility in aqueous media. Silicone-containing polymers possess the additional advantage of having only low brittleness and in some cases being plastically deformable, which is advantageous especially for uses on the skin. Increased gas perviousness can also be desirable in such uses, for which silicone-based materials are particularly suitable on account of their high gas permeability.

For these reasons, there have already been attempts to use silicones as vehicles for active substance. However, as described in J. Controlled Release 1990, 12, 121-132, the active substances need to be present in the silicone matrix in particle form in the systems known to date. Active substance release is then based on the active substance particles swelling in water, resulting in the formation of pores or channels in the silicone polymer through which the active substance may be released into an aqueous environment.

A disadvantage of this approach is that the controlled release of active substance from the matrix depends on the swelling capacity of the active substance particles used in the presence of water and consequently may be controlled only to a limited degree, for example through the size of the active substance particles. US 2013/0172419 has, moreover, demonstrated that release of active substances from pure silicone materials does not occur to any significant extent.

US 2013/0172419 describes composite materials derived from silicone polymers having ionic phenylsulfonate moieties, which are able to absorb active substances from aqueous solution and release them again evenly into an aqueous medium. The production of these materials is, however, a multistep process and therefore costly, particularly as it requires toxic reagents (α-methylstyrene, chlorosulfonic acid) in excess, which need to be removed during processing.

For medical uses in particular, it is necessary to remove such contamination completely. In addition, a composite system containing silicate structures is absolutely necessary for such materials, since ionomeric silicones are not strong enough on their own. The silicate reinforcement does, however, cause a disadvantageous decrease in controlled-release capability.

### SUMMARY OF THE INVENTION

The invention relates to a process, for the release of an active, substance (A) , in which a shaped body (S) containing a composition which comprises a silicone-containing polymer (P) and an active substance (A) is admixed with water, resulting in the release of active substance (A) from the composition (C), wherein the silicon-containing polymer (P) contains one or more radicals bearing at least one amino acid group, bonded to silicon via a carbon atom of a hydrocarbon(oxy) group.

The silicone-containing polymer (P) contains at least one siloxane unit of the general formula I and optionally one or more units of the general formula II

R¹_{b}(X)_{c}SiO_{[4}−(b+c)]/2 (I),

R²ₐSiO_{(4-a)/2} (II),

in which
R¹ and R² independently represent hydrogen or an unbranched, branched or cyclic, saturated or unsaturated alkyl group having 1 to 20 carbon atoms, aryl group or aralkyl group, wherein individual nonadjacent methylene units may be replaced by —O—, —CO—, —COO—, —OCO— or —OCOO—, —S— or NR^{x} groups or by an oxyalkylene group of the general formula (—O—CH₂—CHR³—)_{d},
R³ represents hydrogen or alkyl,
R^{x} represents hydrogen or a C₁-C₁₀ hydrocarbon radical that is unsubstituted or substituted with substituents selected from —CN and halogen,
X represents a radical bearing at least one amino acid unit and attached to the silicon atom via a carbon atom, having the general formula

—Y —NR⁴—(CH₂)ₑ—CR⁵R⁶—COOM,
M represents hydrogen, metal or an ammonium moiety NR¹⁰₄_{⁺},
R¹⁰ independently represents hydrogen or C₁-C₁₂ alkyl, aryl or aralkyl,
R⁴ represents hydrogen or a straight-chain, branched or cyclic, saturated or unsaturated alkyl group having 1 to 20 carbon atoms or aryl group or aralkyl group, wherein individual nonadjacent methylene units may be replaced by —O—, —CO—, —COO—, —OCO— or —OCOO—, —S— or NR^{x} groups or by an oxyalkylene group of the general formula (—O—CH₂—CHR³—)_{d},
R⁵ and R⁶ independently represent hydrogen or straight-chain, branched or cyclic, saturated or unsaturated alkyl group having 1 to 20 carbon atoms or aryl groups or aralkyl groups, wherein individual nonadjacent methylene units may be replaced by —O—, —CO—, —COO—, —OCO— or —OCOO—, —S— or NR^{x} groups, wherein R⁵ or R⁶ may be attached to R⁴ ,
Y represents a straight-chain, branched, cyclic, saturated or mono- or polyunsaturated C₁ to C₁₀₀ alkylene radical attached to the organosilicon compound via a carbon atom, in which individual carbon atoms may be replaced by oxygen, nitrogen or sulfur atoms,
a has the values 0, 1, 2 or 3,
b has the values 0, 1, or 2,
c has the values 1, 2 or 3
d has integer values from 1 to 100.
b+c have the values 1, 2, 3 or 4, and
f has integer values from 0 to 50.

The invention. also relates to a shaped body (S) containing the composition (C) that comprises the silicone-containing polymer (P) and the active substance (A).

It has now been surprisingly found that the silicone-containing polymers (P), which bear amino acid moieties, are very well suited as solid vehicle materials for the controlled release of active substances (A) (controlled-release process) and do not have the abovementioned disadvantages. In particular, the silicone-containing polymers (P) surprisingly show good permeability to water, which is of benefit throughout the controlled-release process. This means that the composition (C) releases active substance (A) in a controlled manner on addition of water or contact therewith.

A further advantage of the process is that amino acids known to be nontoxic and biocompatible may be used for the production of the polymeric solid.

The silicone-containing polymer (P) is preferably a solid at 20° C. and 1 bar.

In particular, the silicone-containing polymer (P) is solid in the range of from −10° C. to 50° and 1 bar.

The amino acid moieties —Y—NR⁴—(CH₂)ₑ—CR⁵R⁶—COOM may be present in various protonation states. Carboxylic acid moieties may be present as the free carboxylic acid or as the carboxylate salt or as a mixture of the two. The amino moiety may be present either as the free amino group or in protonated form, as an ammonium moiety or as a mixture of the two.

R¹ and R² independently represent hydrogen or an unbranched, branched or cyclic, saturated or unsaturated alkyl group having 1 to 6 carbon atoms or a benzyl group or phenyl group, wherein nonadjacent methylene units may be replaced by nitrogen atoms or oxygen atoms or by an oxyalkylene group of the general formula (—O—CH₂—CHR³—)_{d}. The radicals R³ are preferably hydrogen or methyl, in particular methyl.

R^{x} preferably represents hydrogen or an unsubstituted hydrocarbon radical.

M preferably represents hydrogen, alkali metal or alkaline earth metal, more preferably alkali metal, especially preferably sodium or potassium, or an ammonium moiety. R¹⁰ preferably represents hydrogen or C₁-C₄ alkyl.

R⁴ preferably represents hydrogen or a straight-chain, branched or cyclic, saturated or unsaturated alkyl group having 1 to 10 carbon atoms or a benzyl or phenyl group, wherein nonadjacent methylene units may be replaced by nitrogen atoms or oxygen atoms or by an oxyalkylene group of the general formula (—O—CH₂—CHR³—)_{d}. R⁴ more preferably represents a C₁-C₆ alkyl group, wherein methylene units may be replaced by oxyalkylene groups of the general formula —O—(CH₂—CHR³—)_{d}, in particular methyl. The radicals R³ are preferably hydrogen or methyl, in particular methyl.

R⁵ preferably represents hydrogen and R⁶ hydrogen or a straight-chain, branched or cyclic, saturated or unsaturated alkyl group having 1 to 10 carbon atoms or aryl group or aralkyl group, in which individual nonadjacent methylene units may be replaced by —O—, —CO—, —COO—, —OCO— or —OCOO—, —S— or NR^{x} groups. In particular, R⁵ hydrogen and R⁶ hydrogen or —CH₃, —CH(CH₃)₂, —CH₂—CH(CH₃)₂, —CH(CH₃)—CH₂—CH₃, —CH₂—OH, —CH₂—CH₂—OH, —CHOH—CH₃, —CH₂—SH, —CH₂—S—S—CH₂—CH(NH₂)COOH, —CH₂—CH₂—S—CH₃, —CH₂—CH₂—CONH₂, —CH₂—CONH₂, CH₂—CH₂—COOH, CH₂—COOH, —CH₂—CH₂—CH₂—NH—CO—NH₂, —CH₂-phenyl, —CH₂-(4-hydroxyphenyl), —CH₂—CH₂—CH₂—CH₂—NH₂, —CH₂—CH₂—CH₂—NH₂, —CH₂—CH₂—CH₂—NH—C(═NH)—NH₂, and —CH₂-(4-imidazolyl), —CH₂-(3-indolyl).

If R⁴ is attached to R⁵ or to R⁶, then the radicals are preferably connected by an alkylene radical, in particular one having 1 to 6 carbon atoms.

Y preferably represents a straight-chain or branched, saturated to C₁ to C₂₀ alkylene radical, in which individual carbon atoms may be replaced by oxygen, nitrogen or sulfur atoms. More preferably, Y represents the moiety (Z)ₑ—CR⁷(OH)—CR⁸R⁹.

Z preferably represents a straight-chain, branched, cyclic, saturated or mono- or polyunsaturated to C₁ to C₁₀₀ alkylene radical attached to the organosilicon compound via a carbon atom, in which individual carbon atoms may be replaced by oxygen atoms. More preferably, Z represents an oxyalkylene radical of the general formula —CH₂—CH₂—CH₂—O—(CH₂—CHR¹¹—O)_{f}—CH₂, in which trig radicals R¹¹ independently represent hydrogen or alkyl, in particular methyl, and f has a value of 0 to 100, preferably 0 to 50, and more preferably 0.

The radicals R⁷, R⁸, and R⁹ independently represent hydrogen or a straight-chain C₁ to C₆ alkyl group, more preferably hydrogen or a straight-chain C₁ to C₃ alkyl group.

The radicals R⁸ and R⁹ also be attached to one another and to the moiety Z via alkylene radicals or oxygen.

d and f each independently represent preferably 0 to 50, more preferably 0 to 10, more preferably 0 to 5, and most preferably the values 0 to 3.

e represents preferably 0 to 10, more preferably 0 to 5, and most preferably 0 to 3.

The silicone polymers (P) used for the controlled release of active substances (A) may be produced in a manner known to those skilled in the art. For example, the following reactions may be employed to attach the amino acids to the silicon:

(a) addition of the amino groups in amino acids to epoxy-functionalized siloxanes,

(b) addition of amino acids containing thiol moieties to vinyl siloxanes,

(c) nucleophilic substitution of chloroalkyl-functionalized siloxanes by the amino group of amino acids,

(d) reaction of anhydride-functionalized siloxanes with amino acids.

The preferred method of attachment is option (a), in which the amino group of the amino acid undergoes addition to an epoxide, moiety present in the silicone polymer. It is also possible for a double addition to take place here, i.e. two silicone radicals may be attached per amino group present. If an amino group contains more than one basic nitrogen-containing group, these may react in identical manner, which means that a total of 2 siloxane radicals may be attached to each amino group present.

The siloxane units and amino acid units may be in different arrangements relative to one another. Taking lysine by way of example, examples of this are shown below:

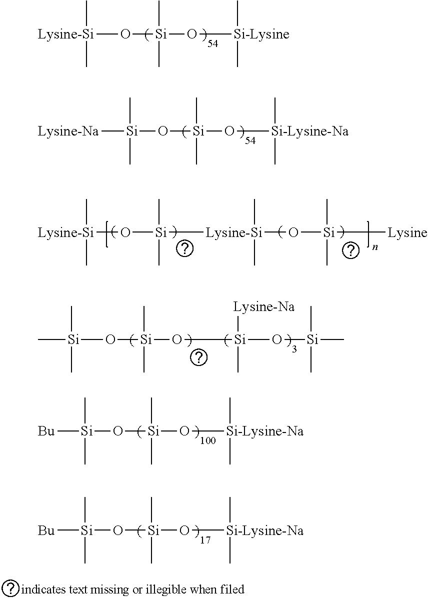

The silicone-containing polymers (P) used for the release of active substances (A) may contain additional polymers. These may form homogeneous or nonhomogeneous mixtures, they may be attached to the polymer (P) covalently or through hydrogen bonds, or they may not be attached at all. The silicone-containing polymers (P) preferably contain these further polymers in proportions of not less than 1 and not more than 95 parts by weight, more preferably in proportions of not less than 5 and not more than 50 parts by weight, most preferably in not less than 10 and not more than 30 parts by weight per 100 parts by weight of siloxane units of the general formula I and II.

### DETAILED DESCRIPTION OF THE PREFERRED EMBODIMENTS

The silicone-containing polymer (P) may contain blocks of siloxane units of the general formula I and no or at least one unit of the general formula II and organic blocks, through which the blocks of siloxane, units are interrupted. The organic blocks may be attached to the blocks siloxane units, for example through urea moieties, urethane moieties or ester groups. The proportion of organic blocks is preferably not less than 2 and not more than 300 parts by weight, more preferably not more than 30 parts by weight, most preferably not more than 10 parts by weight per 100 parts by weight of siloxane units of the general formula I and II.

The polymers (P) used for the controlled release of active substances (A) may be crosslinked or uncrosslinked.

The composition (C) may contain further substances that are present either as solids or in dissolved form.

Examples of solids that may be used are fillers, for example colloidal silica, silicates, zeolites or carbon-based fillers such as carbon black.

The composition (C) may also contain liquids or liquid mixtures as additives. These components may accelerate, or reduce the release of the active substance (A).

The proportion of liquid additives is preferably not less than 0.01 and not more than 1000 parts by weight, more preferably not less than 0.1 and not more than 100 parts by weight, and in particular not less than 1 and not more than 10 parts by weight per 100 parts by weight of siloxane units of the general formula I and II.

Preferred liquid additives are water, organic liquids or mixtures thereof, for example. further siloxanes, esters, monohydric or polyhydric alcohols, ethers, and/or hydrocarbons.

The release of one or more active substances (A) is achieved for example through contact of the composition (C) with damp materials and surfaces or aqueous liquids or indirectly through exposure to water via the gas phase.

Within the shaped body (S) there may be further layers between the composition (C) and the water-containing medium that permit the transport of water in either liquid or gaseous form. The transport of water through these layers in liquid or gaseous form may take place through pores or through channels.

Examples of shaped bodies (S) are granules, sticks, flakes, films, materials containing cavities, for example, membranes, foams or honeycomb structures.

Active substances (A) in this context are preferably substances that exert an effect on biological systems.

These include, drug substances (active pharmaceutical ingredients), hormones, vitamins, trace elements, crop protection agents, pheromones, phytohormones, fragrances, enzymes, coenzymes or antibodies, chelating agent, metals or metal ions, light absorbers, antioxidants, and pigments.

These active substance groups are defined and classified for example Römpp, Chemie Lexikon Online, Thieme, Stuttgart 2015.

The shaped body (S) may be produced, for example, by admixing a solution of the polymer (P) with the active substance (A) in the desired amount and then preferably concentrating the resulting mixture by evaporation. This forms composition (C). This ensures, for example, a uniform distribution of the active. substance (A) in the polymeric matrix of the silicone-containing polymer (P).

Another possibility is to bring the shaped body (S) containing the polymer (P) into contact with liquid, for example water, or with a water containing mixture, that contains the active substance (A). The active substance (A) can in this manner diffuse from the liquid into the polymer (P). This likewise forms composition (C).

After concentration by evaporation or after contact with liquid, the solid composition (C) may if necessary be comminuted to produce smaller shaped bodies (S). The solution of the polymer (P) containing the active substance (S) may also be transferred to molds and concentrated by evaporation.

The release, of active substance (A) occurs preferably over periods of not less than 1 minute to not more than 5 years, more preferably over periods of not less than 1 h to not more than 1 year, most preferably over periods of not less than 1 day to 6 months.

The release of active substance (A) occurs preferably at temperatures of not less than −20° C. aid not more. than +200° C., more preferably of not less than 0° C. and not more. than 100° C., most preferably of not less than +10° C. and not more than +50° C.

The release of active substance (A) occurs preferably at a pressure between not less than 0.1 mbar to not more than 50 bar, more preferably not less than 100 mbar to not more than 20 bar, especially preferably at not less than 0.9 bar to 10 bar.

The release of active substance (A) may also be controlled through the pH or change in the pH or also electrically through change in the potential.

In the examples that follow, unless otherwise stated in each case, all amounts and percentages shown are based on weight and all temperatures are 20° C.

The meanings of all abovementioned symbols in the abovementioned formulae are in each case independent of one another. The silicon atom is tetravalent in all formulae.

### EXAMPLE 1 (PREPARATION OF THE POLYMERIC SILICONE-CONTAINING MATERIAL (P))

50 g of a silicone polymer terminally functionalized with glycidoxypropyl groups and having a molecular weight of approximately 900 is dissolved in 75 ml of methanol and added to a solution of 10 g of lysine in 260 g of methanol. The clear polymer solution is boiled under reflux for 9 hours, filtered, and concentrated to a solid content of approximately 40%.

### EXAMPLE 2 (INTRODUCTION OF THE ACTIVE SUBSTANCE (A) INTO THE POLYMER MATRIX)

352 mg of nicotinic acid is dissolved in 6.0 g of the polymer solution prepared according to example 1. The active substance-containing polymer solution is poured into a Teflon mold. The solvent is evaporated to yield a clear polymer film containing nicotinic acid.

### EXAMPLE 3

489 mg of the polymer film containing 22.9 mg of nicotinic acid obtained according to example 2 is dissolved in. 3 ml of water at 20° C. After specified times t, the amount of nicotinic acid dissolved in water is determined by NMR spectroscopy.

The values shown below in Table 1 are obtained:

TABLE 1

| | mg nicotinic |
|---|---|
| t (hours) | acid in H₂O |
| 0 | 0 |
| 1 | 0.0369 |
| 4 | 0.0885 |
| 24 | 0.233 |
| 96 | 0.462 |

1.-13. (canceled)

14. A shaped body comprising at least one composition comprising a silicone-containing polymer (P) and an active substance (A),
wherein the silicone-containing polymer (P) contains at least one siloxane unit of the formula I and optionally one or more units of the formula II
R¹_{b}(X)_{c}SiO_{[4}−(b+c)]/2 (I),
R²ₐSiO_{(4-a)/2} (II),
in which
R¹ and R² independently represent hydrogen or an unbranched, branched or cyclic, saturated or unsaturated alkyl group having 1 to 20 carbon atoms, an aryl group, or an aralkyl group, wherein individual nonadjacent methylene units are optionally replaced by —O—, —CO—, —COO—, —OCO— or —OCOO—, —S— or NR^{x} groups or by an oxyalkylene group of the formula (—O—CH₂—CHR³—)_{d},
R³ represents hydrogen or alkyl,
R^{x} represents hydrogen or a C₁-C₁₀ hydrocarbon radical that is unsubstituted or substituted with —CN or halogen,
X represents a radical bearing at least one amino acid unit and attached to a silicon atom of the silicon-containing polymer via a carbon atom, and having the formula
—Y—NR⁴—(CH₂)ₑ—CR⁵R⁶—COOM,
wherein M represents hydrogen, metal or an ammonium moiety NR¹⁰₄_{⁺},
R¹⁰ independently represents hydrogen or C₁-C₁₂, alkyl, aryl or aralkyl,
R⁴ represents hydrogen or a straight-chain, branched or cyclic, saturated or unsaturated alkyl group having 1 to 20 carbon atoms or aryl group or aralkyl group, wherein individual nonadjacent methylene units are optionally replaced by —O—, —CO—, —COO—, —OCO— or —OCOO—, —S— or NR^{x} groups or by an oxyalkylene group of the general formula (—O—CH₂—CHR³—)_{d},
R⁵ and R⁶ independently represent hydrogen or straight-chain, branched or cyclic, saturated or unsaturated alkyl groups having 1 to 20 carbon atoms or aryl groups or aralkyl groups, wherein individual nonadjacent methylene units are optionally replaced by —O—, —CO—, —COO—, —OCO— or —OCOO—, —S— or NR^{x} groups, wherein R⁵ or R⁶ are optionally attached to R⁴,
Y represents a straight-chain, branched, cyclic, saturated or mono- or polyunsaturated C₁ to C₁₀₀ alkylene radical attached to the organosilicon compound via a carbon atom, in which individual carbon atoms are optionally replaced by oxygen, nitrogen or sulphur atoms,
a is 0, 1, 2 or 3,
b is 0, 1, or 2,
c is 1, 2 or 3
d is an integer from 1 to 100.
b+c is 1, 2, 3 or 4, and
f is an integer from 0 to 50.

15. The shaped body of claim 14, in which R¹ and R² independently represent an unbranched, branched, or cyclic, saturated or unsaturated alkyl group having 1 to 6 carbon atoms.

16. The shaped body of claim 14, in which M is independently from hydrogen, sodium, potassium, or ammonium moiety.

17. The shaped body of claim 15, in which M is independently from hydrogen, sodium, potassium, or ammonium moiety.

18. The shaped body of claim 14, in which e represents values from 0 to 5.

19. The shaped body of claim 15, in which e represents values from 0 to 5.

20. The shaped body of claim 16, in which cerepresents values from 0 to 5.

21. The shaped body of claim 14, in which the shaped body contains further polymers other than silicone-containing polymer (P).

22. The shaped body of claim 14, in which the silicone-containing polymer (P) contains blocks of siloxane units of the formula and optionally one or more units of the formula II, and organic blocks which interrupt blocks of siloxane units.

23. The shaped body of claim 14, in which the active substance (A) exerts an effect on a biological system.

24. The shaped body of claim 14, in which the release of the active substance (A) occurs over a period of not less than 1 minute to not more than 5 years.

25. The shaped body of claim 13, wherein the shaped body has the form of granules, sticks, flakes, films or materials containing cavities.

26. The shaped body of claim 25, wherein the films or materials containing cavities are membranes, foams, or honeycomb structures.

27. A process for the production of a shaped body of claim 14, in which a solution of the silicone-containing polymer (P) is admixed with the active substance (A) in the desired amount and the resulting mixture is concentrated by evaporation to form the composition.

28. The process of claim 27, wherein the composition is a solid composition which has been comminuted after concentration by evaporation.

29. A process for the release of an active substance (A) from a shaped body of claim 14, wherein the composition is brought into contact with a damp material, damp surfaces, aqueous liquids, or directly with water in a gas phase.

30. The shaped body of claim 14, which supplies a medicament.
